(19) United States
(12) Patent Application Publication
(10) Pub. No.: US 2018/0344565 A1
(43) Pub. Date: Dec. 6, 2018

(54) MULTI-CHAMBERED MALE SEXUAL STIMULATION DEVICE

(71) Applicant: Brian Sloan, Skokie, IL (US)

(72) Inventor: Brian Sloan, Skokie, IL (US)

(21) Appl. No.: 15/995,081

(22) Filed: May 31, 2018

Related U.S. Application Data
(60) Provisional application No. 62/513,434, filed on May 31, 2017.

(51) Int. Cl. A61H 19/00

(52) U.S. Cl. CPC A61H 19/32; A61F 5/41; A61H 2201/0153; A61H 2201/0103

(57) ABSTRACT

A multi-chambered sheath-type male masturbation device that contains different orifice shapes and stimulation patterns molded into each chamber to provide a complex stimulation profile based on perpendicular pressure inward toward the penis in patterns molded into the sleeve combined with axial movement along the penis. The multiple chambers allow the user to change the type stimulation profile during use by switching chambers.

### CROSS-REFERENCE TO RELATED APPLICATIONS

The present application claims priority to U.S. provisional patent application Ser. No. 62/513,434, titled “Pluri-Chamber Simulating Stimulator” and filed on May 31, 2017, the entire specification of which is incorporated herewith by reference.

### BACKGROUND

### Field of the Art

The present invention is in the field of devices for sexual stimulation, and more particularly in the field of devices for male masturbation.

### Discussion of the State of the Art

Sheath-type devices for male sexual stimulation typically provide only one type of sensation. To obtain a different type of sensation, another device must be purchased and used separately. Such devices are generally not configurable to adjust the suction, pressure, texture, and patterns of stimulation to maximize the user's enjoyment. Additionally, they do not allow for a convenient method of removing the fragile internal sheath for cleaning purposes.

What is needed is a male masturbation device that provides multiple stimulation options in the same device, and provides controls and configurations for adjusting suction, pressure, texture, and patterns of stimulation, and allows for convenient removal of, and protection of, the fragile internal sheath for cleaning.

### SUMMARY

Accordingly, the inventor has conceived, and reduced to practice, a multi-chambered sheath-type male masturbation device that allows the user to choose which type of stimulation he prefers at a given moment, allows the user to adjust suction and pressure during usage, allows for customization of the sensation by exchanging the internal soft sleeve in one or more of the chambers, and allows for convenient removal of the internal sheath contained inside a semi-rigid liner for cleaning purposes. There are several distinct benefits of this device. The device may be used without the need for electrical power, allowing the user to use the device in any location. The device provides a complex stimulation profile based on perpendicular pressure inward toward the penis in complex patterns molded into the sleeve combined with axial movement along the penis. The flexible sleeve is contained within a semi-rigid liner, which protects the sleeve against damage. The device is designed such that the entire semi-rigid liner can be easily removed with the sleeve inside, allowing convenient cleaning of the device without risk of damage to the flexible sleeve.

In a preferred embodiment, the device is a small handheld unit with three tube-shaped chambers arranged side-by-side in linear fashion. The housing of the device is made from a rigid material such as molded plastic. The housing is hinged to allow removal of the semi-rigid liner with the sleeve unit inside, and is closed with screws on the opposite side of the housing. Inside the housing is a semi-rigid liner encompassing each chamber that assists in providing pressure and snugness against the penis. Inside the liner is a removable inner sleeve made of a soft, flexible, textured material into which a different orifice shape and a pattern of stimulation ridges has been molded for each chamber. Stimulation is achieved by inserting the erect penis into the orifice of one of the sleeves and manually moving the entire device in a reciprocal motion along the shaft of the penis. Stimulation is enhanced by the textured materials chosen for the inner sleeve, the orifice shape, and by the pattern of stimulation molded into the sleeve. At any time, the penis may be withdrawn from one chamber and inserted into a different chamber for a different sensation. Dials at the end of each chamber on both sides of the device adjust air valves, which controls the amount of suction provided by the device. Air is vented perpendicularly to the axis of the chamber.

In a second preferred embodiment, the device is a small handheld unit with three tube-shaped chambers arranged side-by-side in linear fashion. The housing of the device is made from a rigid material such as molded plastic. The housing is hinged to allow removal of the semi-rigid liner with the sleeve unit inside, and is closed with plastic on the opposite side of the housing. Inside the housing is a semi-rigid liner encompassing each chamber that assists in providing pressure and snugness against the penis. Inside the liner is a removable inner sleeve made of a soft, flexible, textured material into which a different orifice shape and a pattern of stimulation ridges has been molded for each chamber. Stimulation is achieved by inserting the erect penis into the orifice of one of the sleeves and manually moving the entire device in a reciprocal motion along the shaft of the penis. Stimulation is enhanced by the textured materials chosen for the inner sleeve, the orifice shape, and by the pattern of stimulation molded into the sleeve. At any time, the penis may be withdrawn from one chamber and inserted into a different chamber for a different sensation. A dial at the end of each chamber on one side of the device adjusts an air valve, which controls the amount of suction provided by the device. Air is vented axially from the end of the sleeve, and perpendicularly to the axis of the chamber through the semi-rigid liner and housing.

In a third preferred embodiment, the tube-shaped chambers are arranged into a cluster, such as a triangle for a three chambered device, a square for four chambered device, etc., in order to make the device easier to manipulate and to facilitate switching chambers during usage. In an aspect of this embodiment, the semi-rigid liner between the housing and sleeve is replaced by an inflatable bladder liner, and a small, manually operated pump is provided to fill or deflate the bladder liner, allowing the user to adjust the pressure and snugness of each chamber during use.

### BRIEF DESCRIPTION OF THE DRAWING FIGURES

The accompanying drawings illustrate several aspects and, together with the description, serve to explain the principles of the invention according to the aspects. It will be appreciated by one skilled in the art that the particular arrangements illustrated in the drawings are merely exemplary, and are not to be considered as limiting of the scope of the invention or the claims herein in any way.

FIG. 1 shows a partially exploded view of an exemplary male sexual stimulation device according to a preferred embodiment.

FIG. 2 shows an exploded, cutaway view of an exemplary aspect of a preferred embodiment, one of multiple chambers surrounded by a semi-rigid liner with a replaceable sleeve of soft, flexible, textured material molded with an orifice and stimulation pattern.

FIG. 3 shows a partially exploded view of an exemplary male sexual stimulation device according to a preferred embodiment, with two parts of the housing attached at the hinges.

FIG. 4 shows an isometric view of an exemplary male sexual stimulation device according to a preferred embodiment, partially assembled with the housing attached at the hinges, the semi-rigid liner inserted into the housing and the sleeve unit inserted into the semi-rigid liner.

FIG. 5 shows an isometric view of an exemplary aspect of a preferred embodiment, a one-piece molded replaceable sleeve unit.

FIG. 6 shows a side view of an exemplary aspect of a preferred embodiment, the one-piece molded replaceable sleeve unit.

FIG. 7 shows a front view of an exemplary aspect of a preferred embodiment, the one-piece molded replaceable sleeve unit.

FIG. 8 shows a cutaway view of an exemplary aspect of a preferred embodiment, an exemplary first sleeve of the one-piece molded replaceable sleeve unit.

FIG. 9 shows a cutaway view of an exemplary aspect of a preferred embodiment, an exemplary second sleeve of the one-piece molded replaceable sleeve unit.

FIG. 10 shows a cutaway view of an exemplary aspect of a preferred embodiment, an exemplary third sleeve of the one-piece molded replaceable sleeve unit.

FIG. 11 shows a sectional view of an exemplary aspect of a preferred embodiment, a semi-rigid intermediate liner enclosing the sleeve in one of multiple chambers.

FIG. 12 shows a cutaway view of an exemplary aspect of a preferred embodiment, a semi-rigid intermediate liner enclosing the sleeve in one of multiple chambers.

FIG. 13 shows a sectional view of an exemplary aspect of a preferred embodiment, the housing enclosing the semi-rigid liner which encloses the sleeve unit for one of multiple chambers.

FIG. 14 shows a cutaway view of an exemplary aspect of a preferred embodiment, the housing enclosing the semi-rigid liner which encloses the sleeve unit for one of multiple chambers.

FIG. 15 shows a partially exploded view of an exemplary male sexual stimulation device according to another preferred embodiment.

FIG. 16 shows a partially exploded view of an exemplary male sexual stimulation device according to a preferred embodiment, with two parts of the housing attached at the hinges.

FIG. 17 shows a sectional view of exemplary aspects of a preferred embodiment, the flexible sleeve, the semi-rigid liner, and the housing.

### DETAILED DESCRIPTION

The inventor has conceived, and reduced to practice, a multi-chambered sheath-type male masturbation device that allows the user to choose which type of stimulation he prefers at a given moment, allows the user to adjust suction and pressure during usage, allows for customization of the sensation by exchanging the internal soft sleeve in one or more of the chambers, and allows for convenient removal of the internal sheath contained inside a semi-rigid liner for cleaning purposes.

The device is unique, in that it allows for multiple different types of sensation in a single, handheld unit. The device may contain any number of different chambers, arranged in one of a plurality of arrangements. For example, in one embodiment, the device could contain three chambers arranged side-by-side in a linear fashion. In another embodiment, the same three chambers could be clustered together in a triangular fashion. A four-chambered device could be arranged in a linear fashion, a square fashion, or in a semi-circle. Adding chambers to the device provides additional stimulation options for a given device, and modifying the arrangement of the chambers allows for different methods of holding and using the device.

According to an embodiment, the outside housing of the device would be made of a rigid material such as molded plastic. It may be textured or have rubberized grips or other coverings to enhance grasping and holding the device during use. Inside the housing is a semi-rigid liner, whose purpose is to contain the sleeve, and provide inward pressure against the penis inside the sleeve. The semi-rigid liner may be removable to allow for removal of the liner with the sleeve unit inside to facilitate cleaning and protect the sleeve unit, as well as to provide options regarding pressure and snugness. Inside the semi-rigid liner is a removable sleeve made of a soft, flexible material. The material from which the sleeve is made may be chosen to provide different firmness and texture.

The removable sleeve may have orifices and patterns of ridges to simulate different types of actual sexual activity, or simply to provide a unique sensation not available elsewhere. A tri-chambered device, for example could have inner sheaths with orifices and patterns designed to simulate vaginal, oral, and anal intercourse, and the user would be free to switch between the three different types of stimulation simply by withdrawing the penis from one chamber and inserting it into a different one. Other types of unique stimulation can be achieved by changing the sleeve material, the shape of the orifice, and the stimulation pattern inside the sleeve.

Through the combination of the semi-rigid liner and molded sleeve unit, the device provides a complex stimulation profile based on perpendicular pressure inward toward the penis in complex patterns molded into the sleeve combined with axial movement along the penis. The pattern of stimulation molded into the sleeve unit is applied to the penis either by moving the device manually along the shaft of the penis or by having the user thrust the penis into the device.

The device may be used without the need for electrical power, allowing the user to use the device in any location. Further, because of the lack of motors, the device operates silently, which may be of significant benefit in certain environments.

The flexible sleeves associated with such devices are made of fragile materials such as thermoplastic elastomer (TPE), which tears easily. To protect against damage to the flexible sleeve, it is contained within a semi-rigid liner, whereby the liner is handled instead of the sleeve and the sleeve is contained to prevent flexing, stretching, and possible tearing. The device is designed such that the entire semi-rigid liner can be easily removed with the sleeve inside, allowing convenient cleaning of the device without risk of damage to the flexible sleeve.

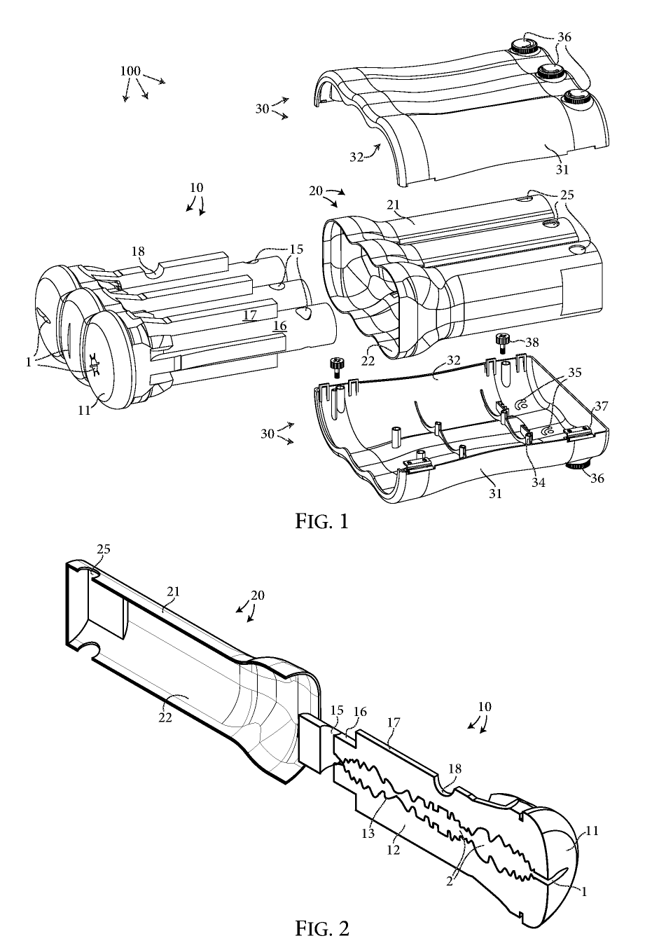

FIG. 1 shows a partially exploded view of an exemplary male sexual stimulation device 100 according to a preferred embodiment. The exterior of the device is a rigid external housing 30, molded from hard plastic or other suitable material 31. On the outside of the housing 30 are dials 36 that allow venting of the sleeve 10 through ports 15 near the end of the sleeve 10 which vent through ports 25 near the end of the semi-rigid liner 20, and finally through the vents 35 in the housing 30. The venting controls the amount of suction provide by each of the sleeves of the sleeve unit 10. Inside the housing 30 are molded brackets 34 that hold the liner 20 and sleeve unit 10 in place. The housing 30 has hinges 37 at one side, allowing the unit to be opened to replace the liner 20, and reclosed using screws 38 on the opposite side of the housing 30. At the front of the housing 30 is a large opening 32, which allows the insertion of the flexible sleeve unit 10. Inside the housing 30 is a semi-rigid liner 20 made of semi-rigid plastic or other suitable material 21 that assists in providing pressure against the penis inside the sleeve 10. At the front of the semi-rigid liner is a large opening 22, into which the sleeve unit 10 is inserted. The sleeve unit 10 is comprised of a plurality of tubular channels 16, each with a rounded top 11 and a different shape of orifice 1. On the outside of each sleeve are guide ridges 17 and indentations 18 that hold the sleeve unit in place inside the housing 30.

FIG. 2 shows an exploded, cutaway view of an exemplary aspect of a preferred embodiment, one of multiple chambers surrounded by a semi-rigid liner 20 with a replaceable sleeve unit 10. The sleeve unit 10 is comprised of a plurality of tubular sleeves 16, each with a rounded top 11 and a different shape of orifice 1. The sleeve unit 10 is made of a soft, flexible, textured material 12 into which a different orifice shape 1 and a pattern of stimulation ridges 13 is molded for each chamber, providing a channels 2 into which the penis is inserted.

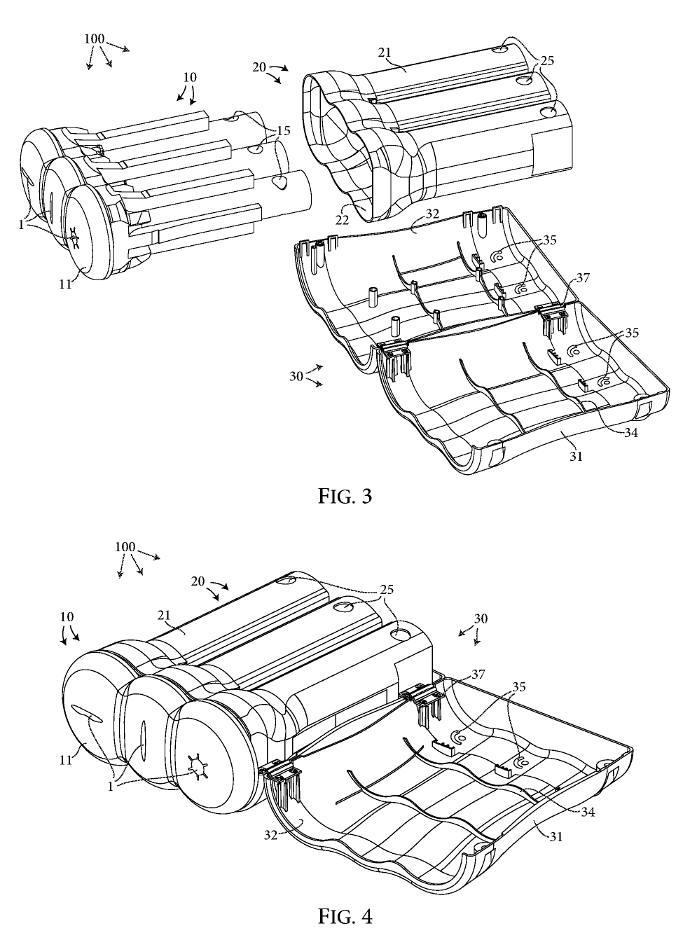

FIG. 3 shows a partially exploded view of an exemplary male sexual stimulation device 100 according to a preferred embodiment, with two parts of the housing 30 attached at the hinges 37. The exterior of the device is a rigid external housing 30, molded from hard plastic or other suitable material 31. On the outside of the housing 30 are dials 36 (shown in FIG. 1) that allow venting of the sleeve 10 through ports 15 near the end of the sleeve 10 which vent through ports 25 near the end of the semi-rigid liner 20, and finally through the vents 35 in the housing 30. The venting controls the amount of suction provide by each of the sleeves of the sleeve unit 10. Inside the housing 30 are molded brackets 34 that hold the liner 20 and sleeve unit 10 in place. The housing 30 has hinges 37 at one side, allowing the unit to be opened to replace the liner 20, and reclosed using screws 38 on the opposite side of the housing 30. At the front of the housing 30 is a large opening 32, which allows the insertion of the flexible sleeve unit 10. Inside the housing 30 is a semi-rigid liner 20 made of semi-rigid plastic or other suitable material 21 that assists in providing pressure against the penis inside the sleeve 10. At the front of the semi-rigid liner is a large opening 22, into which the sleeve unit 10 is inserted. The sleeve unit 10 is comprised of a plurality of tubular channels 16, each with a rounded top 11 and a different shape of orifice 1. On the outside of each sleeve are guide ridges 17 and indentations 18 that hold the sleeve unit in place inside the housing 30.

FIG. 4 shows an isometric view of an exemplary male sexual stimulation device 100 according to a preferred embodiment, partially assembled with the housing 30 attached at the hinges 37, the semi-rigid liner 20 inserted into the housing 30 and the sleeve unit 10 inserted into the semi-rigid liner 20. The exterior of the device is a rigid external housing 30, molded from hard plastic or other suitable material 31. On the outside of the housing 30 are dials 36 (shown in FIG. 1) that allow venting of the sleeve 10 through ports 15 (shown in FIG. 1) near the end of the sleeve 10 which vent through ports 25 near the end of the semi-rigid liner 20, and finally through the vents 35 in the housing 30. The venting controls the amount of suction provide by each of the sleeves of the sleeve unit 10. Inside the housing 30 are molded brackets 34 that hold the liner 20 and sleeve unit 10 in place. The housing 30 has hinges 37 at one side, allowing the unit to be opened to replace the liner 20, and reclosed using screws 38 on the opposite side of the housing 30. At the front of the housing 30 is a large opening 32, which allows the insertion of the flexible sleeve unit 10. Inside the housing 30 is a semi-rigid liner 20 made of semi-rigid plastic or other suitable material 21 that assists in providing pressure against the penis inside the sleeve 10. At the front of the semi-rigid liner is a large opening 22 (shown in FIG. 1), into which the sleeve unit 10 is inserted. The sleeve unit 10 is comprised of a plurality of tubular channels 16 (shown in FIG. 1), each with a rounded top 11 and a different shape of orifice 1. On the outside of each sleeve are guide ridges 17 (shown in FIG. 1) and indentations 18 (shown in FIG. 1) that hold the sleeve unit in place inside the housing 30.

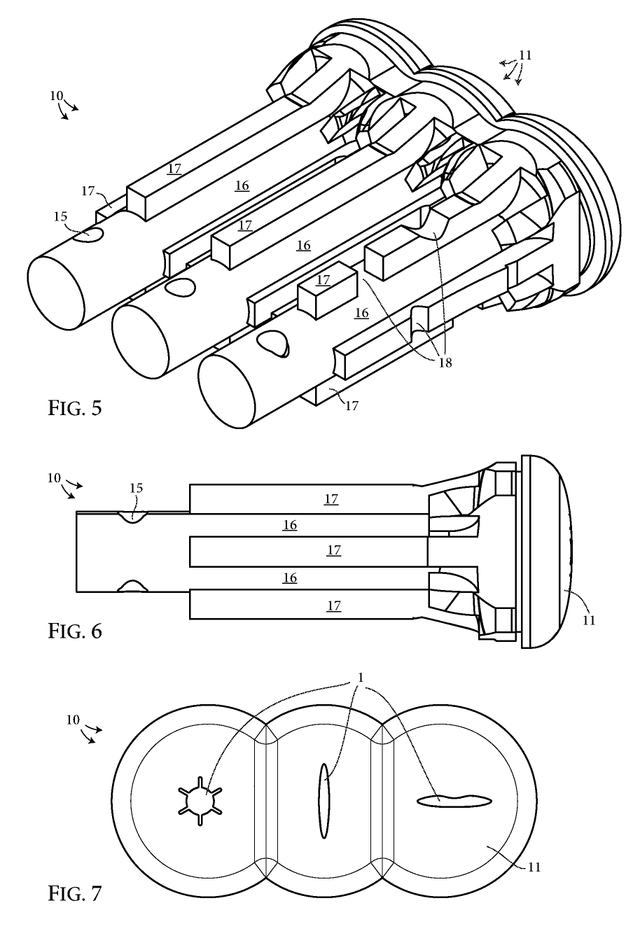

FIG. 5 shows an isometric view of an exemplary aspect of a preferred embodiment, a one-piece molded replaceable sleeve unit 10. The sleeve unit 10 is comprised of a plurality of tubular channels 16, each with a rounded top 11 and a different shape of orifice 1 (shown in FIG. 1). On the outside of each sleeve are guide ridges 17 and indentations 18 that hold the sleeve unit in place inside the housing 30. The sleeve unit 10 is made of a soft, flexible, textured material 12 into which a different orifice shape 1 and a pattern of stimulation ridges 13 is molded for each chamber, providing a channels 2 into which the penis is inserted.

FIG. 6 shows a side view of an exemplary aspect of a preferred embodiment, the one-piece molded replaceable sleeve unit 10. The sleeve unit 10 is comprised of a plurality of tubular channels 16, each with a rounded top 11 and a different shape of orifice 1 (shown in FIG. 1). On the outside of each sleeve are guide ridges 17 and indentations 18 (shown in FIG. 1) that hold the sleeve unit in place inside the housing 30 (shown in FIG. 1).

FIG. 7 shows a front view of an exemplary aspect of a preferred embodiment, the one-piece molded replaceable sleeve unit 10. The sleeve unit 10 is comprised of a plurality of tubular channels 16 (shown in FIG. 1), each with a rounded top 11 and a different shape of orifice 1.

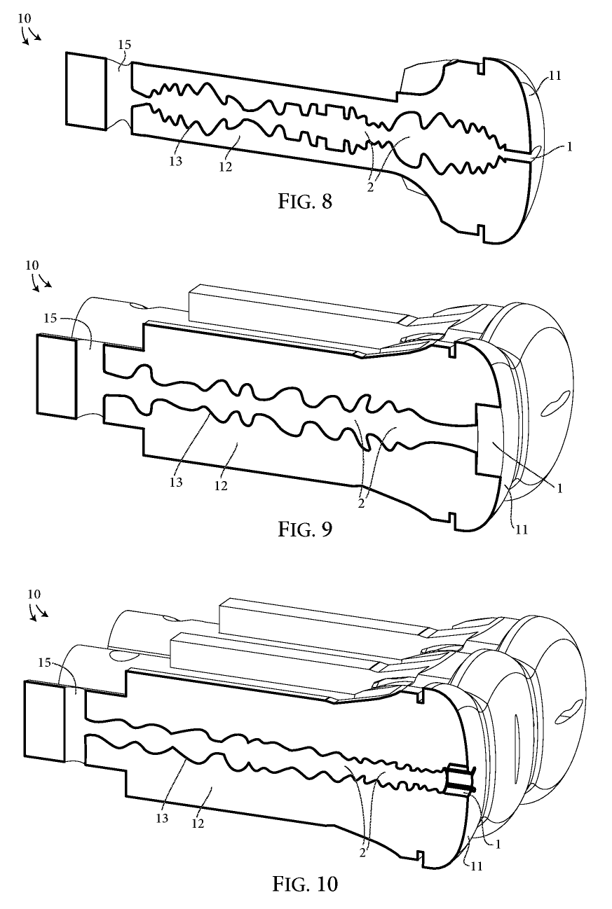

FIG. 8 shows a cutaway view of an exemplary aspect of a preferred embodiment, an exemplary first sleeve of the one-piece molded replaceable sleeve unit 10. Each sleeve 16 has a rounded top 11 and a different shape of orifice 1. Each sleeve 16 is made of a soft, flexible, textured material 12 into which a different orifice shape 1 and a pattern of stimulation ridges 13 has been molded, providing a channel 2 into which the penis is inserted.

FIG. 9 shows a cutaway view of an exemplary aspect of a preferred embodiment, an exemplary second sleeve of the one-piece molded replaceable sleeve unit 10. Each sleeve 16 has a rounded top 11 and a different shape of orifice 1. Each sleeve 16 is made of a soft, flexible, textured material 12 into which a different orifice shape 1 and a pattern of stimulation ridges 13 has been molded, providing a channel 2 into which the penis is inserted.

FIG. 10 shows a cutaway view of an exemplary aspect of a preferred embodiment, an exemplary third sleeve of the one-piece molded replaceable sleeve unit 10 containing three sleeves of soft, flexible, textured material, each molded with a different orifice and stimulation pattern. Each sleeve 16 has a rounded top 11 and a different shape of orifice 1. Each sleeve 16 is made of a soft, flexible, textured material 12 into which a different orifice shape 1 and a pattern of stimulation ridges 13 has been molded, providing a channel 2 into which the penis is inserted.

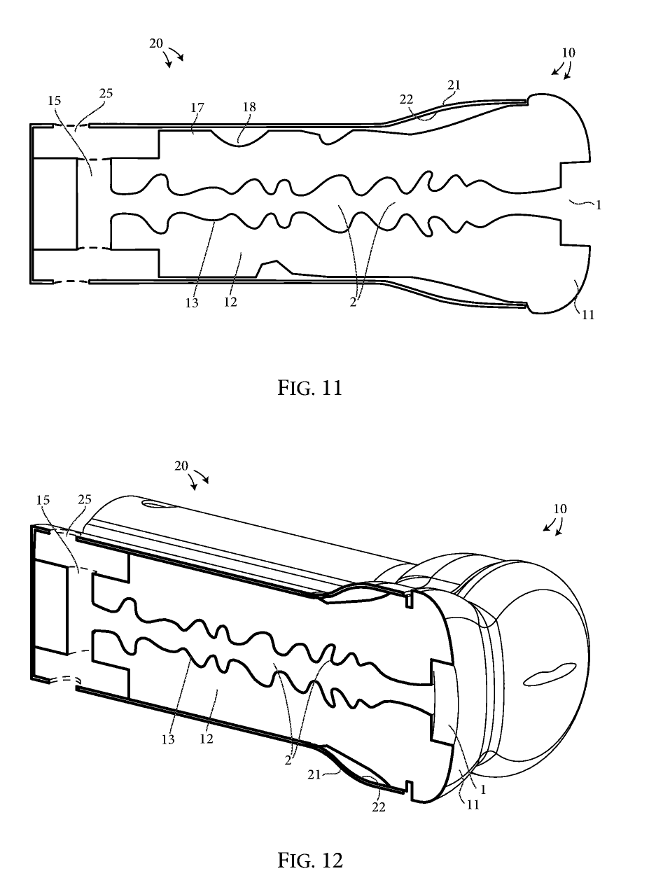

FIG. 11 shows a sectional view of an exemplary aspect of a preferred embodiment, a semi-rigid intermediate liner 20 enclosing the sleeve in one of multiple chambers. The semi-rigid liner 20 is made of semi-rigid plastic or other suitable material 21 that assists in providing pressure against the penis inside the sleeve 10. The liner 20 contains vents 25 that connect with vents in the sleeve 15 to control the amount of suction provided in a particular sleeve. At the front of the semi-rigid liner is a large opening 22, into which the sleeve unit 10 is inserted. Each sleeve 16 (shown in FIG. 1) has a rounded top 11 and a different shape of orifice 1. Each sleeve 16 (shown in FIG. 1) is made of a soft, flexible, textured material 12 into which a different orifice shape 1 and a pattern of stimulation ridges 13 has been molded, providing a channel 2 into which the penis is inserted.

FIG. 12 shows a cutaway view of an exemplary aspect of a preferred embodiment, a semi-rigid intermediate liner 20 enclosing the sleeve in one of multiple chambers. The semi-rigid liner 20 is made of semi-rigid plastic or other suitable material 21 that assists in providing pressure against the penis inside the sleeve 10. The liner 20 contains vents 25 that connect with vents in the sleeve 15 to control the amount of suction provided in a particular sleeve. At the front of the semi-rigid liner is a large opening 22, into which the sleeve unit 10 is inserted. Each sleeve 16 (shown in FIG. 1) has a rounded top 11 and a different shape of orifice 1. Each sleeve 16 (shown in FIG. 1) is made of a soft, flexible, textured material 12 into which a different orifice shape 1 and a pattern of stimulation ridges 13 has been molded, providing a channel 2 into which the penis is inserted.

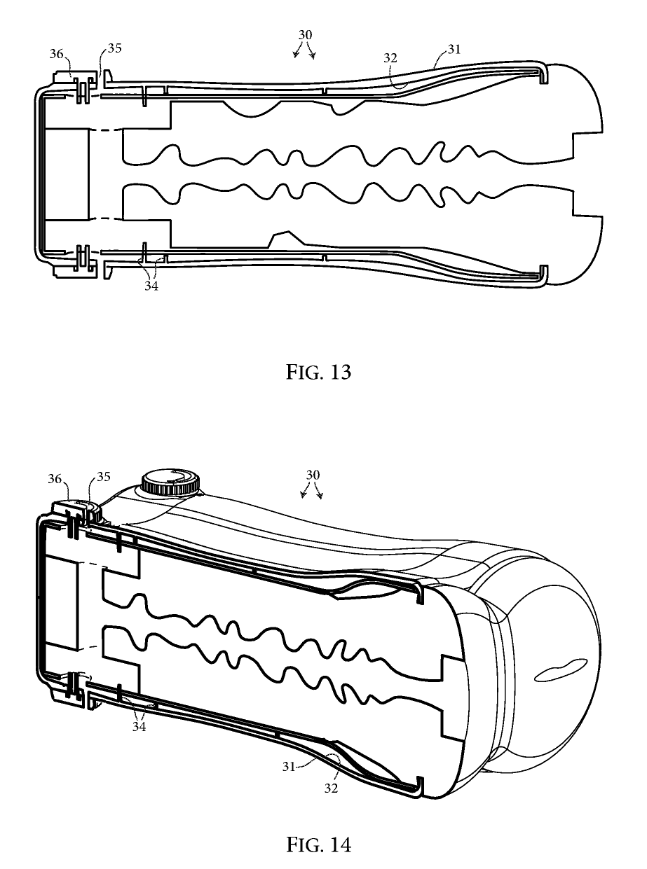

FIG. 13 shows a sectional view of an exemplary aspect of a preferred embodiment, the housing enclosing the semi-rigid liner 20 (shown in FIG. 1) which encloses the sleeve unit 10 (shown in FIG. 1) for one of multiple chambers. The housing 30 is molded from hard plastic or other suitable material 31. At the front of the housing 30 is a large opening 32, which allows the insertion of the flexible sleeve unit 10. On the outside of the housing 30 are dials 36 that allow venting of the sleeve 10 through ports 15 (shown in FIG. 1) near the end of the sleeve 10 which vent through ports 25 (shown in FIG. 1) near the end of the semi-rigid liner 20, and finally through the vents 35 in the housing 30. The venting controls the amount of suction provide by each of the sleeves of the sleeve unit 10. Inside the housing 30 are molded brackets 34 that hold the liner 20 and sleeve unit 10 in place.

FIG. 14 shows a cutaway view of an exemplary aspect of a preferred embodiment, the housing enclosing the semi-rigid liner 20 which encloses the sleeve unit 10 for one of multiple chambers. The housing 30 is molded from hard plastic or other suitable material 31. At the front of the housing 30 is a large opening 32, which allows the insertion of the flexible sleeve unit 10 (shown in FIG. 1). On the outside of the housing 30 are dials 36 that allow venting of the sleeve 10 through ports 15 (shown in FIG. 1) near the end of the sleeve 10 which vent through ports 25 (shown in FIG. 1) near the end of the semi-rigid liner 20 (shown in FIG. 1), and finally through the vents 35 in the housing 30. The venting controls the amount of suction provide by each of the sleeves of the sleeve unit 10. Inside the housing 30 are molded brackets 34 that hold the liner 20 and sleeve unit 10 in place.

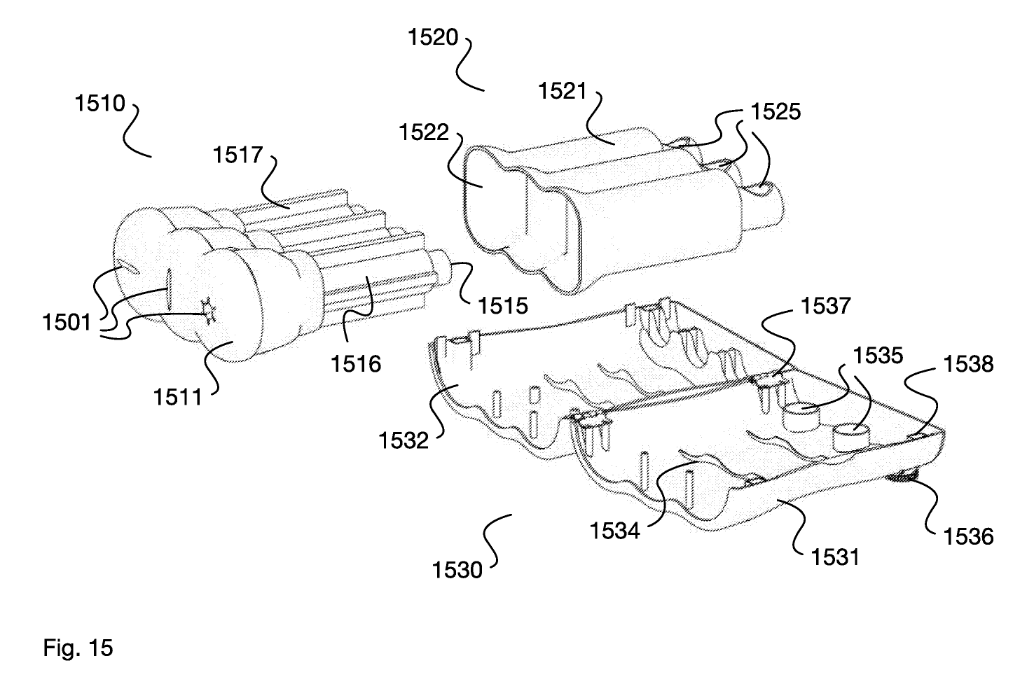

FIG. 15 shows a partially exploded view of an exemplary male sexual stimulation device according to a second preferred embodiment. This embodiment simplifies certain aspects of design and manufacture, and places the porting axially instead of perpendicularly, which strengthens the structure of the porting system. The exterior of the device is a rigid external housing 1530, molded from hard plastic or other suitable material 1531. On one side of the outside of the housing 1530 are dials 1536 that allow venting of the sleeve 1510 through ports 1515 at the axial end the end of the sleeve 1510 which vent through perpendicular ports 1525 near the end of the semi-rigid liner 1520, and finally through the vents 1535 in the housing 1530. The venting controls the amount of suction provide by each of the sleeves of the sleeve unit 1510. Inside the housing 1530 are molded brackets 1534 that hold the liner 1520 and sleeve unit 1510 in place. The housing 1530 has hinges 1537 at one side, allowing the unit to be opened to remove the liner 1520 with the sleeve unit 1510 enclosed inside for cleaning, and allowing the unit to be reclosed using plastic tabs 1538 on the opposite side of the housing 1530. At the front of the housing 1530 is a large opening 1532, which allows the insertion of the flexible sleeve unit 1510. Inside the housing 1530 is a semi-rigid liner 1520 made of semi-rigid plastic or other suitable material 1521 that assists in providing pressure against the penis inside the sleeve 1510. At the front of the semi-rigid liner is a large opening 1522, into which the sleeve unit 1510 is inserted. The sleeve unit 1510 is comprised of a plurality of tubular channels 1516, each with a rounded top 1511 and a different shape of orifice 1501. On the outside of each sleeve are guide ridges 1517 that hold the sleeve unit in place inside the housing 1530.

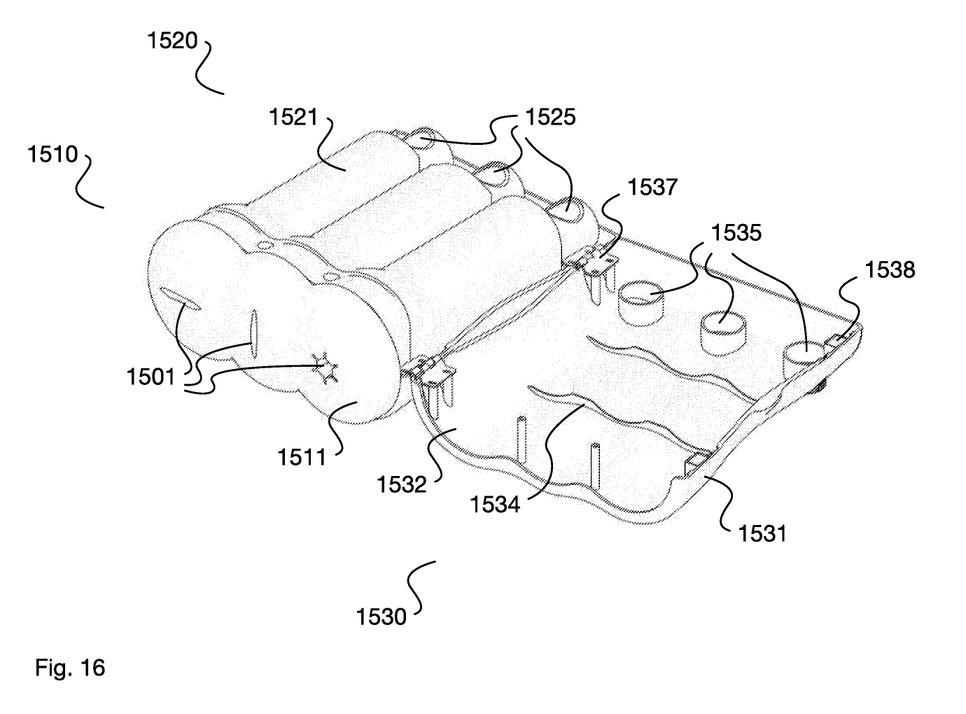

FIG. 16 shows a partially exploded view of an exemplary male sexual stimulation device according to the second preferred embodiment, with two parts of the housing 1530 attached at the hinges 1537. The exterior of the device is a rigid external housing 1530, molded from hard plastic or other suitable material 1531. On one side of the outside of the housing 1530 are dials 1536 that allow venting of the sleeve 1510 through ports 1515 at the axial end the end of the sleeve 1510 which vent through perpendicular ports 1525 near the end of the semi-rigid liner 1520, and finally through the vents 1535 in the housing 1530. The venting controls the amount of suction provide by each of the sleeves of the sleeve unit 1510. Inside the housing 1530 are molded brackets 1534 that hold the liner 1520 and sleeve unit 1510 in place. The housing 1530 has hinges 1537 at one side, allowing the unit to be opened to remove the liner 1520 with the sleeve unit 1510 enclosed inside for cleaning, and allowing the unit to be reclosed using plastic tabs 1538 on the opposite side of the housing 1530. At the front of the housing 1530 is a large opening 1532, which allows the insertion of the flexible sleeve unit 1510. Inside the housing 1530 is a semi-rigid liner 1520 made of semi-rigid plastic or other suitable material 1521 that assists in providing pressure against the penis inside the sleeve 1510. At the front of the semi-rigid liner is a large opening 1522, into which the sleeve unit 1510 is inserted. The sleeve unit 1510 is comprised of a plurality of tubular channels 1516, each with a rounded top 1511 and a different shape of orifice 1501. On the outside of each sleeve are guide ridges 1517 that hold the sleeve unit in place inside the housing 1530.

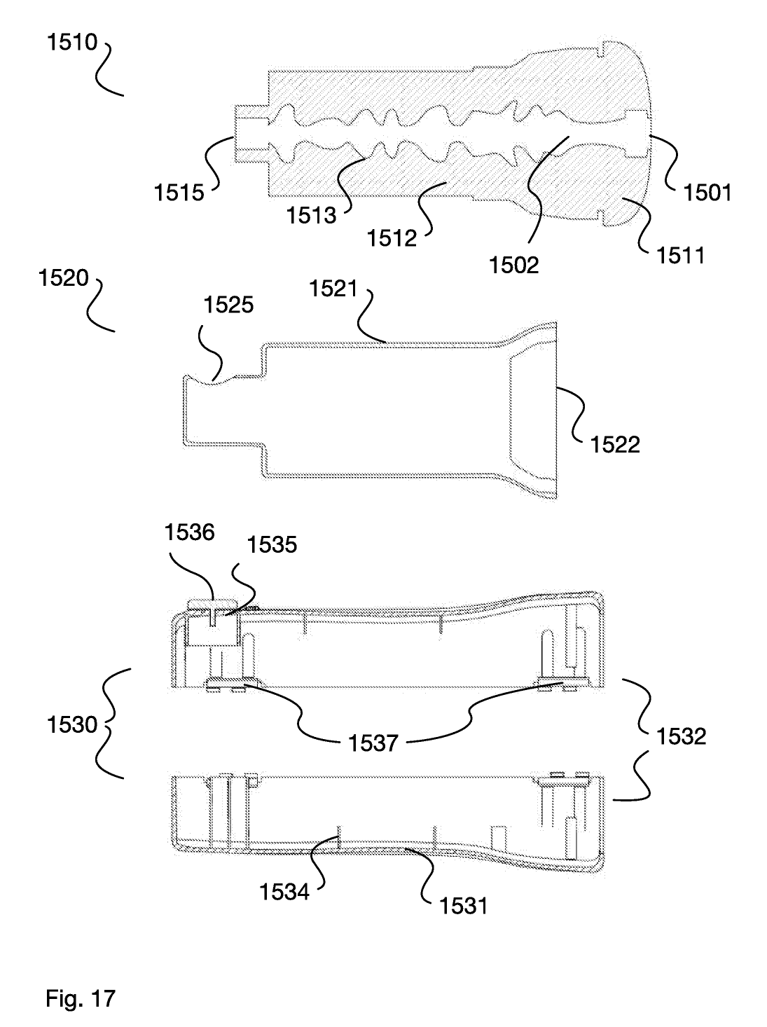

FIG. 17 shows a sectional view of exemplary aspects of the second preferred embodiment, the flexible sleeve unit 1510, the semi-rigid liner 1520, and the housing 1530. The exterior of the device is a rigid external housing 1530, molded from hard plastic or other suitable material 1531. On one side of the outside of the housing 1530 are dials 1536 that allow venting of the sleeve 1510 through ports 1515 at the axial end the end of the sleeve 1510 which vent through perpendicular ports 1525 near the end of the semi-rigid liner 1520, and finally through the vents 1535 in the housing 1530. The venting controls the amount of suction provide by each of the sleeves of the sleeve unit 1510. Inside the housing 1530 are molded brackets 1534 that hold the liner 1520 and sleeve unit 1510 in place. The housing 1530 has hinges 1537 at one side, allowing the unit to be opened to remove the liner 1520 with the sleeve unit 1510 enclosed inside for cleaning, and allowing the unit to be reclosed using plastic tabs 1538 (shown in FIG. 15) on the opposite side of the housing 1530. At the front of the housing 1530 is a large opening 1532, which allows the insertion of the flexible sleeve unit 1510. Inside the housing 1530 is a semi-rigid liner 1520 made of semi-rigid plastic or other suitable material 1521 that assists in providing pressure against the penis inside the sleeve 1510. At the front of the semi-rigid liner is a large opening 1522, into which the sleeve unit 1510 is inserted. The sleeve unit 1510 is comprised of a plurality of tubular channels 1516, each with a rounded top 1511 and a different shape of orifice 1501. On the outside of each sleeve are guide ridges 1517 that hold the sleeve unit in place inside the housing 1530.

One or more different aspects may be described in the present application. Further, for one or more of the aspects described herein, numerous alternative arrangements may be described; it should be appreciated that these are presented for illustrative purposes only and are not limiting of the aspects contained herein or the claims presented herein in any way. One or more of the arrangements may be widely applicable to numerous aspects, as may be readily apparent from the disclosure. In general, arrangements are described in sufficient detail to enable those skilled in the art to practice one or more of the aspects, and it should be appreciated that other arrangements may be utilized and that structural, logical, software, electrical and other changes may be made without departing from the scope of the particular aspects. Particular features of one or more of the aspects described herein may be described with reference to one or more particular aspects or figures that form a part of the present disclosure, and in which are shown, by way of illustration, specific arrangements of one or more of the aspects. It should be appreciated, however, that such features are not limited to usage in the one or more particular aspects or figures with reference to which they are described. The present disclosure is neither a literal description of all arrangements of one or more of the aspects nor a listing of features of one or more of the aspects that must be present in all arrangements.

Headings of sections provided in this patent application and the title of this patent application are for convenience only, and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible aspects and in order to more fully illustrate one or more aspects. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the aspects, and does not imply that the illustrated process is preferred. Also, steps are generally described once per aspect, but this does not mean they must occur once, or that they may only occur once each time a process, method, or algorithm is carried out or executed. Some steps may be omitted in some aspects or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other aspects need not include the device itself.

Techniques and mechanisms described or referenced herein will sometimes be described in singular form for clarity. However, it should be appreciated that particular aspects may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various aspects in which, for example, functions may be executed out of order from that shown or discussed, including substantially concurrently or in reverse order, depending on the functionality involved, as would be understood by those having ordinary skill in the art.

What is claimed is:

1. A male sexual stimulation device comprising:
an external housing made of rigid material, and containing at least one tubular chamber, a vent and venting dial for each chamber perpendicular to the axis of the chamber, and capable of being opened to remove an intermediate liner;
a removable intermediate liner made of a semi-rigid material, contained inside the housing and surrounding, at least partially, each of the tubular chambers, and containing a vent for each chamber perpendicular to the axis of the chamber; and
an internal sleeve made of a soft, flexible material, contained within the intermediate liner, molded to allow a channel containing a different orifice and stimulation pattern axially along the inside of the sleeve for each chamber, and containing a vent for each chamber perpendicular to the axis of the chamber;
wherein as an erect penis is inserted into, or removed from, a channel in the sleeve, pressure is applied inwardly against the penis in the stimulation pattern molded into the channel the sleeve along.

2. The device of claim 1, wherein the vent in the sleeve for each chamber is at the closed end of the sleeve and along the axis of the chamber instead of perpendicular to the axis of the chamber.

3. The device of claim 1, wherein the intermediate liner is an inflatable bladder along the inside of each tubular chamber that may be pressurized to assist in providing pressure and snugness against the penis contained in the sleeve.
